(19) United States
(12) Patent Application Publication
(10) Pub. No.: US 2020/0238233 A1
(43) Pub. Date: Jul. 30, 2020

(54) A METHOD OF MIXING FLUID FLOWING IN A RACEWAY CHANNEL AND RACEWAY CHANNEL THEREFOR

(71) Applicant: University of Cape Town, Cape Town (ZA)

(72) Inventors: Matthew Armstrong Burke, Cape Town (ZA); Susan Therese Largier Harrison, Cape Town (ZA)

(21) Appl. No.: 16/756,687

(22) PCT Filed: Oct. 30, 2018
(86) PCT No.: PCT/IB2018/058484

§ 371 Date: Apr. 16, 2020

(30) Foreign Application Priority Data
Oct. 30, 2017 (GB) 1717837.7

(51) Int. Cl. B01F 5/10; B01F 5/06; C02F 3/32; C12M 1/02; A01G 33/00

(52) U.S. Cl. CPC B01F 5/102; B01F 5/061; C02F 3/322; B01F 2215/0422; A01G 33/00; B01F 2005/0622; C12M 27/00

(57) ABSTRACT

A method of mixing fluid flowing in a raceway channel is provided which includes directing the fluid to flow over a ramp which extends across the raceway channel. The ramp has a leading surface which is inclined at between about 5° and 45° upwardly to a top edge, and a trailing surface which extends vertically downwardly from the top edge. The height of the ramp is selected so that the top edge is lower than the level of the fluid in the raceway channel.

### CROSS-REFERENCE(S) TO RELATED APPLICATIONS

This application claims priority from United Kingdom patent application number 1717837.7 filed on 30 Oct. 2017, which is incorporated by reference herein.

### FIELD OF THE INVENTION

This invention relates to a method of mixing fluid flowing in a channel, more particularly a raceway channel used in the production of algae.

### BACKGROUND TO THE INVENTION

The raceway pond is the most commonly used reactor for the growth of algae, due to low capital costs, low operating costs and ease of installation. These are channels configured in an endless loop, typically in an oval shape reminiscent of an automotive or horse racing track, with a pump or paddlewheel configured to cause the water or fluid to flow continuously around the raceway. Currently one of the largest difficulties with all photobioreactors is the low rate of carbon dioxide mass transfer into the system. This limits algal growth and productivity as the photosynthetic rate is directly affected by usage carbon concentration. The algae within these systems only obtain carbon from the dissolved inorganic carbon, whether in the form of carbon dioxide, bicarbonate or carbonate, and hence sufficient carbon dioxide mass transfer is one of the most important design parameters for any photobioreactor, to ensure that the growth rate is optimised. This is particularly evident in raceway ponds as they have a lower mass transfer rate than other photobioreactors and hence are typically mass transfer limited. Various methods have been proposed to improve the mass transfer within raceway ponds, including sparging with carbon dioxide, trapping the carbon dioxide within the raceways using covers, using carbon dioxide permeable membranes, using carbonation columns outside the reactor, and combining airlift and raceway designs.

Other methods propose baffles and similar flow altering devices within algal raceway channels. In An Investigation into Delta Wing Vortex Generators as a Means of Increasing Algae Biofuel Raceway Vertical Mixing Including an Analysis of the Resulting Turbulence Characteristics (Godfrey, 2012) it is proposed to fit “delta wings” into a raceway to improve the mixing. These “delta wings” are triangular, wing-like plates fitted at an angle to the normal fluid flow within the raceway and are meant to creating turbulence and mixing. No mention is made of improving the mass transfer rate.

US 2010/0178686 discloses a raceway having sets of inclined translucent tubes through which fluid flows under gravity and in which paddle wheels are used to displace the fluid from a lower trough up an incline to an elevated trough. This is not a conventional system and during flow in the tubes there will be limited or no carbon dioxide mass transfer. It may also be impractical to move the fluid from a lower level to a higher level up a slop using a paddle wheel.

These prior art methods produce variable results and many contribute significantly to the capital investment or operational costs. As a result there is a need for a system which improves mass transfer into the raceway ponds without significantly adding to the capital or operating costs.

The preceding discussion of the background to the invention is intended only to facilitate an understanding of the present invention. It should be appreciated that the discussion is not an acknowledgment or admission that any of the material referred to was part of the common general knowledge in the art as at the priority date of the application.

### SUMMARY OF THE INVENTION

In accordance with this invention there is provided a method of mixing fluid flowing at a substantially constant level in a raceway channel which includes directing the fluid to flow over a ramp which extends across the raceway channel and has a leading surface which terminates in a top edge and has an upward incline extending at least partway along its length, and a trailing surface which extends vertically downwardly from the top edge, and characterised that the height of the ramp is selected so that the top edge is lower than the level of the fluid in the raceway channel

Further features of the invention provide for the height of the top edge of the ramp to preferably be about one critical depth below the level of the fluid in the raceway channel; for the leading surface to include a level section between the upward incline and the top edge; and for there to be a further upward incline between the level section and the top edge.

Still further features of the invention provide for the leading surface to be inclined at an angle of between about 5° and 45° from the bottom surface of the raceway channel; and for the leading surface to preferably be inclined at an angle of between about 7° and 30° from the bottom surface, more preferably between about 7° and 15°, most preferably between about 7° and 10°.

Yet further features of the invention provide for the dimensions of the ramp to be selected to obtain E₀=0.5 to 3.3, where E₀, is calculated as:

$${E}_{0} = \frac{tan ( α )}{\sqrt{\frac{{H}_{∞}}{{L}_{∞}}}}$$

where α is the angle of inclination of the leading surface, H_{∞} is wave height of the fluid movement in the channel prior to the ramp, and L_{∞} is the wavelength of the fluid movement in the channel prior to the ramp.

The invention also includes a mixing element for a raceway channel in which a fluid flows and in which the level of the fluid in the channel is at a substantially constant level, comprising a ramp which is configured to extend, in use, across the channel and has a leading surface which terminates in a top edge and has an upward incline extending at least partway along its length, and a trailing surface which extends vertically downwardly from the top edge, and characterised in that the height of the ramp is selected so that the top edge is operatively lower than the level of the fluid in the channel.

Further features of the invention provide for the height of the ramp to be selected to be, in use, about one critical depth below the level of the fluid in the raceway channel; for the leading surface to include a level section between the upward incline and the top edge; and for there to be a further upward incline between the level section and the top edge.

Still further features of the invention provide for the leading surface to be inclined at an angle of between 5° and 45° from the bottom surface of the raceway channel in use; and for the leading surface to preferably be inclined at an angle of between about 7° and 30° from the bottom surface, more preferably between about 7° and 15°, most preferably between about 7° and 10°.

The invention still further provides a raceway channel which forms an endless loop and has a pair of sides extending from a bottom characterised in that it includes a mixing element substantially as defined above.

An embodiment of the invention will now be described, by way of example only, with reference to the accompanying drawings.

### BRIEF DESCRIPTION OF THE DRAWINGS

In the drawings:

FIG. 1 is a top plan view of a raceway pond; and

FIG. 2 is a sectional elevation of part of the raceway channel in FIG. 1 showing a mixing element.

FIG. 3 is a sectional elevation of part of a raceway channel showing a second embodiment of a mixing element.

FIG. 4 is a sectional elevation of part of a raceway channel showing a third embodiment of a mixing element.

### DETAILED DESCRIPTION WITH REFERENCE TO THE DRAWINGS

A method of mixing fluid flowing in a raceway channel is provided. The raceway channel forms a pond, or raceway pond, for algal growth and typically contains a fluid consisting of water, algae and nutrients. The fluid is made to circulate about the raceway channel by means of a paddlewheel, pump or other suitable device. The channel may have a flat bottom with substantially upright sides, but may have any suitable cross-sectional shape and size.

The method of mixing the fluid flowing in the raceway channel includes directing the fluid to flow over a ramp which extends across the raceway channel. The ramp thus extends between the sides of the raceway channel to prevent fluid from bypassing the ramp or flowing between the ramp and the sides of the channel.

The ramp has a leading surface which terminates in a top edge and has an upward incline extending at least partway along its length. The upward incline may extend or commence from the bottom of the channel. The ramp further includes a trailing surface which extends substantially vertically downwardly from the top edge.

The leading surface may include a level section, or plateau, between the upward incline and the top edge. The leading surface may include a further upward incline between the level section, or plateau, and the top edge such that the leading surface includes a first upward incline followed by a plateau or level section and then a second upward incline.

More than one level section may also be provided in the leading surface and these may be accompanied by corresponding upward inclines.

The leading surface may be inclined at an angle of between about 5° and 45° from the bottom surface of the raceway channel. The leading surface may preferably be inclined at an angle of between about 7° and 30° from the bottom surface, more preferably between about 7° and 15°, most preferably between about 7° and 10°.

The angle of the incline may vary along the length of the leading surface. The leading surface may thus have, for example, a curved, either convex or concave, shape. Where the leading surface has a level section and more than one upward incline, each upward incline may be at a different angle.

The height of the ramp is be selected so that the top edge is lower than the level of the fluid in the raceway channel. The level of fluid may be selected to be the substantially constant or average level or the level of fluid it is desired to maintain in the raceway channel. It will be appreciated that the raceway channel will typically be designed to operate at a certain or predetermined fluid level and that this fluid level is sought to be maintained. It is level of the fluid which is typically used to select the height of the ramp.

The height may be selected to be about one critical depth below the level of the fluid in the raceway channel. The critical depth (D_{c}) is that depth at which the flow is at its minimum energy with respect to the bottom of the channel. The depth of the fluid stream at critical flow can be calculated using Error! Reference source not found., where Q is the volumetric flow rate and W is the channel width, and hence this is the typical water depth over the top of a weir.

$${D}_{c} = \sqrt[3]{\frac{{Q}^{2}}{g \cdot {W}^{2}}} \qquad Equation 1$$

One critical depth may be this depth below the level or height of the fluid plus or minus approximately 10%. In this specification “about one critical depth” shall mean the calculated depth (D_{c}) below the level or height of the fluid plus or minus approximately 10%.

In nature, waves produce large amounts of turbulence and mass transfer. This is especially true of breaking waves. The angle of inclination of the or each upward incline of the leading edge of the ramp is preferably be selected to produce waves as the fluid flows over the top edge of the ramp. In producing waves in the fluid mass transfer may be greatly enhanced.

The surf similarity parameter, E₀, predicts whether a wave forms a spilling or plunging breaker and may be useful in calculating an optimal angle of inclination. The surf similarity parameter is calculated using Equation 2 and depends on the beach slope, α, wave height, H_{∞}, and wavelength, L_{∞}:

$${E}_{0} = \frac{tan ( α )}{\sqrt{\frac{{H}_{∞}}{{L}_{∞}}}} \qquad Equation 2$$

For spilling breakers E₀=0-0.5, and for plunging breakers E₀=0.5-3.3. It is preferred to obtain a crashing wave, or plunging breaker, using the ramp in the raceway channel and the surf similarity parameter may assist in calculating the dimensions of the ramp to obtain this.

Thus, the dimensions of the ramp could be selected to obtain E₀=0.5 to 3.3, according to Equation 2, where α is the angle of inclination of the leading surface, H_{∞} is wave height of the fluid movement (open-water wave) produced by the paddlewheel (that is, the height of the fluid movement produced by the paddlewheel in the channel section prior to the ramp) and, L_{∞} is the wavelength of the fluid movement (open-water wave) produced by the paddlewheel (that is, the wavelength of the fluid movement produced by the paddlewheel in the channel section prior to the ramp). H_{∞} and L_{∞} are measured in the channel prior to the start of the ramp. Where the ramp has more than one upward inclination, α is the angle of inclination of the last upward inclination, that immediately before the trailing edge.

The optimal slope inclination is dependent on the flowrate, as well as culture density, width to depth ratio, and the like, and thus inclination should be selected to be appropriate for a particular flowrate so that a plunging wave regime is achieved.

The ramp has the further advantage that it results in the fluid being shallower as it passes over the ramp and thus promotes exposure of the fluid, and particularly the algae therein, to light. There is thus a greater chance for a greater proportion of the fluid to be exposed to light as it passes over the ramp than during normal flow within the channel.

The provision of a level section, or plateau, in the leading surface of the ramp provides an extended pathway over which the fluid is shallower than in the remainder of the channel and hence has a greater exposure to light for an extended period of time. This may in turn result in enhanced growth of the algae in the fluid as they are able to store this energy and then utilise it when there is no CO₂ limitation.

In designing the length of the plateau consideration should be given to ensure that the algae are not over exposed to light which could cause stress, or that the additional friction would reduce momentum and negatively impact entrainment caused by the turbulence.

Provision could be made for enhanced or high intensity lighting over the ramp to further take advantage of the greater overall exposure of the fluid to light.

There is also provided a mixing element for use in a method of mixing fluid flowing in a raceway channel substantially as described above. The fluid has a level which is substantially constant, and is typically maintained at such a level. The mixing element includes a ramp which is dimensioned to extend across the channel, between its sides, from the bottom of the channel.

The ramp has a leading surface which terminates in a top edge and has an upward incline extending at least partway along its length. The upward incline may extend or commence from the bottom of the channel. The ramp further includes a trailing surface which extends substantially vertically downwardly from the top edge.

The leading surface may include a level section, or plateau, between the upward incline and the top edge. The leading surface may include a further upward incline between the level section, or plateau, and the top edge such that the leading surface includes a first upward incline followed by a plateau or level section and then a second upward incline.

More than one level section may also be provided in the leading surface and these may be accompanied by corresponding upward inclines. Thus, the leading surface may include multiple upward inclines and multiple level sections or plateaus.

The lowermost end of the leading surface conforms to and extends along the bottom of the channel between the sides. Fluid is thus prevented from flowing between the ramp and the bottom of the channel.

The leading surface may have an incline of between 5° and 45° from the bottom surface of the raceway channel in use, preferably an incline of between 7° and 30°, more preferably between about 7° and 15°, most preferably between about 7° and 10°.

The angle of the incline may vary along the length of the leading surface. The leading surface may thus have, for example, a curved, either convex or concave, shape. Where the leading surface has a level section and more than one upward incline, each upward incline may be at a different angle.

The height of the ramp is be selected so that the top edge is lower than the level of the fluid in the raceway channel. The height may be selected to preferably be about one critical depth below the level of the fluid in the raceway channel.

The mixing element may be formed integrally with the raceway channel or may be provided as a separate unit which is secured within a raceway channel. Where provided as a separate unit, the mixing element can be made from any suitable material. For example, it may be made of a plastics material, a composite material or a sheet metal such as aluminium. The mixing element may be made in such a way as to permit the user to trim its edges to provide a desired fit within a raceway channel.

It is further envisaged that the mixing element may be constructed to enable a user to adjust the angle of the incline of the or each upward incline so as to be able to obtain optimal turbulence for the given raceway channel dimensions and fluid constitution and flow rate.

The invention still further provides a raceway channel which includes a mixing element substantially as defined above.

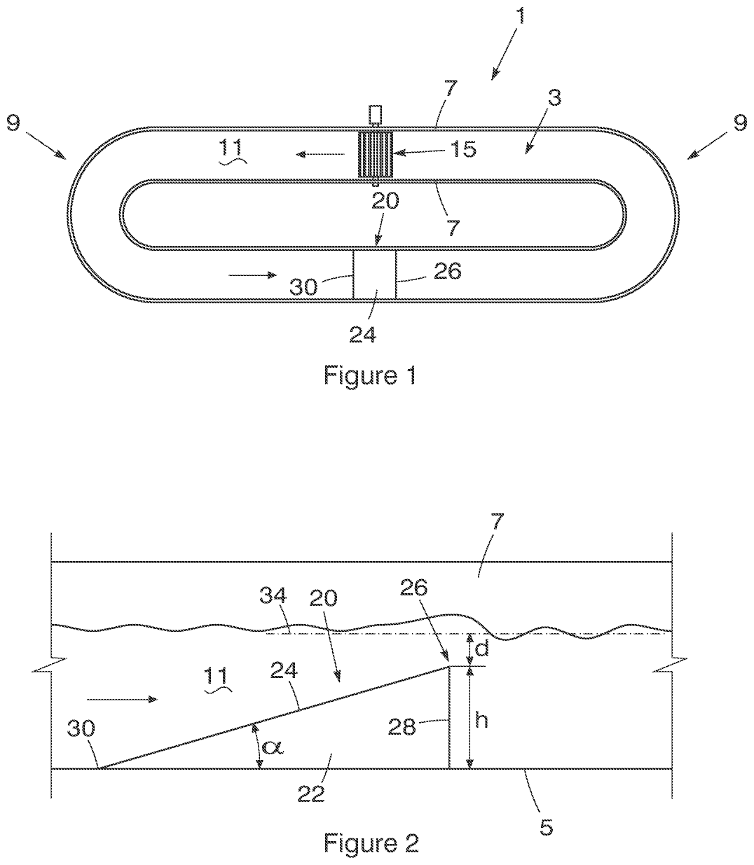

One example of a raceway pond (1) is shown in FIGS. 1 and 2 and includes an elongate, oval raceway channel (3) forming an endless loop. The raceway channel (3) is, in this embodiment, constructed at a laboratory scale from a transparent plastics material and has a flat bottom (5) and substantially upright sides (7). The channel (3) is 150 mm wide and 1000 mm in length between the curved ends (9) each of which has an outer radius of curvature of 250 mm and an inner radius of curvature of 100 mm. The channel (3) has a height of 300 mm and is filled to a height of 200 mm with water (11). A motorised paddlewheel (15) is provided in the raceway channel (3) midway between the ends (9) and is operable to drive the water (11) in the channel so that it flows continuously around the loop.

Thus far, a raceway pond of substantially conventional configuration has been described.

A mixing element (20) is provided in the raceway channel (3) to assist in mixing the fluid (11) flowing therein. In this embodiment the mixing element (20) is provided in the channel (3) opposite the paddlewheel (15). Referring also to FIG. 2, the mixing element (20) includes a ramp (22) which extends across the channel (3), between its sides (7), from the bottom (5) of the channel. The ramp (22) has a leading surface (24) which is inclined upwardly to a top edge (26). A trailing surface (28) extends substantially vertically downwardly from the top edge (26) to the bottom (5) of the channel (3).

The lowermost end (30) of the leading surface (24) conforms to and extends along the bottom (5) of the channel (3) between the sides (7). Similarly, the sides (32) of the ramp (22) abut the sides (7) of the channel (3) along their length. The fluid (11) is thus prevented from flowing between the ramp (22) and the bottom (5) or sides (7) of the channel (3) and is directed to flow over the ramp (22).

The leading surface (24) has, in this embodiment, an incline (a) of 30° as measured from the bottom surface (5) of the raceway channel (3).

The height (h) of the ramp (22) is, in this embodiment, selected so that the top edge (26) is lower than the level (34) of the fluid (11) in the raceway channel (3). It will be appreciated that the level of the fluid may fluctuate to a degree during operation of the raceway pond but that normal operation will typically require a predetermined level to be maintained or at least aimed for.

Further according to this embodiment, the height (h) is selected so that the top edge (26) is about one critical depth (d) below the level (34) of the fluid (11) in the raceway channel (3). The critical depth is that depth at which the flow is at its minimum energy with respect to the bottom of the channel. In this embodiment one critical depth is approximately 20% of the level or height of the fluid plus or minus approximately 10%.

In this embodiment the mixing element (20) is made from a sheet of a plastics material, such as thermoplastic acrylic resin or polystyrene, which is preformed into shape and then secured in the channel (3) using a suitable adhesive sealant.

In use, the leading surface (24) of the mixing element (20) causes a region of fast-moving liquid to be formed near the top end (26) of the ramp (22) which immediately, and turbulently, falls into a slower moving region beyond the trailing surface (28). This results in the creation of breaking of waves and mixing. This mixing can also be considered similar to the flow of liquid over a weir which produces a “hydraulic jump” and associated standing waves just downstream of the end of the weir. The fluid or water flow over the top of a submerged weir is typically critical, indicating that the fluid flow has the minimum specific energy for that particular volumetric flow rate. The critical depth can be calculated using Error! Reference source not found. above. The hydraulic jump, and associated waves, produce significant turbulence in this region of the channel, with very good mixing and some level of entrainment.

For trial purposes, the above mixing element (20) was tested and compared to a further mixing element (not shown) having a leading surface inclined at 7° to the bottom (5) and having the same height (h). Mixing time studies within the raceway channel (3) using both mixing elements were conducted using the pulsed injections of concentrated salt solution that were tracked using conductivity measurements. The visual mixing was also determined using phenolphthalein solution that changed between a clear and pink solution with the addition of concentrated acid or base solutions. These visual tests allowed for the turbulent regions and any dead zones to be identified. The results are shown in Table 1 below.

TABLE 1

| | | | | | Percentage |
|---|---|---|---|---|---|
| | | | Circu- | Percentage | improvement |
| Mixing | | Mixing | lation | improvement | in mixing |
| Element | K_{L}a (s⁻¹) | time (s) | time (s) | in K_{L}a (%) | time (%) |
| None | 0.000177 | 154 | 28 | — | — |
| 7° incli- | 0.000319 | 57 | 35 | 80% | 63% |
| nation | | | | | |
| 30° incli- | 0.000277 | 109 | 33 | 56% | 29% |
| nation | | | | | |

The mixing tests revealed that mixing elements produce significant amounts of turbulence, mixing and surface renewal in the region immediately following the trailing surface. This is in comparison to the normal raceway which only experiences turbulent mixing near the paddlewheel and the fluid flow is almost laminar for the rest of the channel. The improved mixing was demonstrated by the fact that the overall mixing time of the original raceway without a mixing element was 154 s, while the mixing elements with leading surface is inclined at 30° and 7° had mixing times of 109 s and 57 s respectively. The longer mixing time for the 30° leading surface, in comparison to the 7° leading surface, was due to a large dead zone which formed directly after the trailing surface and resulted in poorer hydrodynamic flow.

The mass transfer obtained using the mixing element was also improved, with a 56% and 80% increase for the 30° and 7° inclinations respectively. The increased mixing and mass transfer did not significantly alter the evaporative losses from the pond. The power required, due to extra frictional and turbulence losses, is less than the power required for sparging or other mass transfer improving methods, although still more than for the raceway pond without a mixing element.

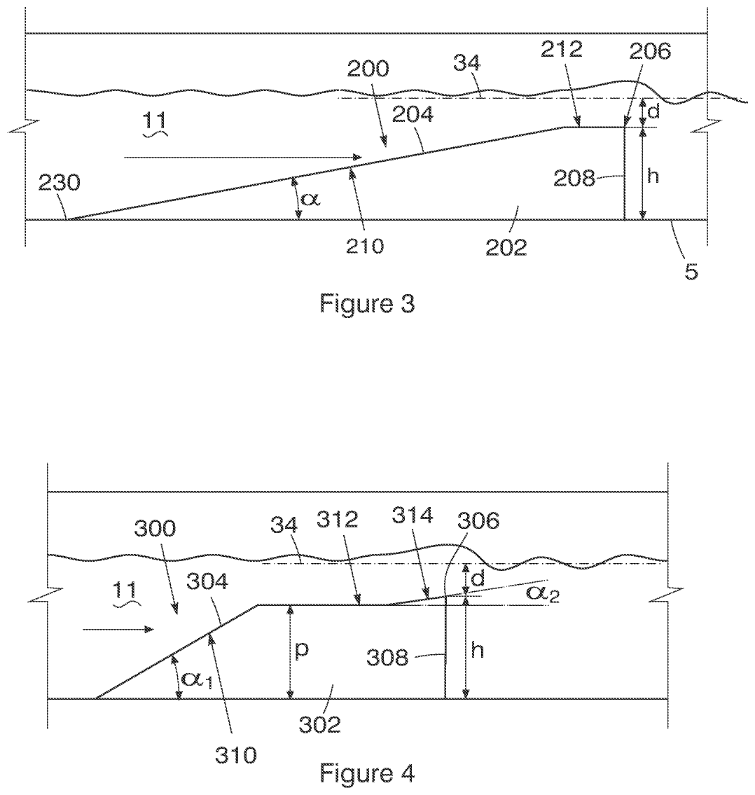

A second embodiment of a mixing element (200) is shown in FIG. 3 and, similarly to the embodiment in FIGS. 1 and 2, includes a ramp (202) which extends across the channel (3), between its sides (7), from the bottom (5) of the channel (3). The ramp (202) has a leading surface (204) which terminates in a top edge (206) and a trailing surface (208) which extends substantially vertically downwardly from the top edge (206) to the bottom (5) of the channel (3).

In this embodiment the ramp (202) is configured to have an upward incline (210) extending partway along its length from the bottom (5) and a level section (212) between the upward incline (210) and the top edge (206).

The leading surface (24) has, in this embodiment, an incline (a) of 10° as measured from the bottom surface (5) of the raceway channel (3). The height (p) of the level section (212) is the same as the height (h) of the top edge (206) of the ramp (200). In this embodiment too, the height (h) of the top edge (206) is lower than the level (34) of the fluid (11) in the raceway channel (3), preferably about one critical depth (d) below the level (34) of the fluid (11).

As with the embodiment in FIGS. 1 and 2, the leading surface (204) of the mixing element (200) causes a region of fast-moving liquid to be formed near the top end (206) of the ramp (202) which immediately, and turbulently, falls into a slower moving region beyond the trailing surface (208) and results in the creation of breaking of waves and mixing. Additionally, the level section (212) of the leading surface (204) results in the fluid (11) being shallower than in the channel (3) as it flows along the length of this section and hence having a greater proportion thereof exposed to light.

The mixing element (200) has the benefit of providing enhanced mass transfer through the turbulence it creates in the fluid, and also enhances exposure of the fluid to light.

A third embodiment of a mixing element (300) is shown in FIG. 4 and is similar to that shown in FIG. 3 in that it includes a ramp (302) which extends across a raceway channel (3), between its sides (7), from the bottom (5) of the channel (3). The leading surface (304) of the ramp (302) terminates in a top edge (306) and a trailing surface (308) extends substantially vertically downwardly from the top edge (306) to the bottom (5) of the channel (3). The ramp (302) is similarly configured to have a first upward incline (310) extending partway along its length and a level section (312) thereafter.

In this embodiment, however, a is provided after the second upward incline (314) thus extends between the level section (312) and the top edge (306).

The height (p) of the level section (312) is less than the height (h) of the top edge (306) of the ramp (300). In this embodiment too, the height (h) of the top edge (306) is lower than the level (34) of the fluid (11) in the raceway channel (3), preferably about one critical depth (d) below the level (34) of the fluid (11).

The angle of inclination (α₁) of the first upward incline (310) also differs, in this embodiment, from the angle of inclination (α₂) of the second upward incline (314). These are selected, in this embodiment such that α₁ is 30° and α₂ is 7°. It will be understood that any suitable angels can be used and that both angles can be the same.

It is envisaged that this embodiment may provide the advantages of good light exposure to all the fluid passing over the level section (312), as well as being able to better control wave formation or turbulence at the top edge, if necessary.

It be further appreciated that many other embodiment of a mixing element exist which fall within the scope of the invention, particularly regarding the configuration and dimensions thereof.

Throughout the specification and claims unless the content requires otherwise the word ‘comprise’ or variations such as ‘comprises’ or ‘comprising’ will be understood to imply the inclusion of a stated integer or group of integers but not the exclusion of any other integer or group of integers.

1. A method of mixing fluid flowing at a substantially constant level in a raceway channel, which method includes directing the fluid to flow over a ramp which extends across the raceway channel and has a leading surface which terminates in a top edge and has an upward incline extending at least partway along its length, and a trailing surface which extends vertically downwardly from the top edge, and wherein the height of the ramp is selected so that the top edge is about one critical depth below the level of the fluid in the raceway channel.

2. (canceled)

3. A method of mixing fluid as claimed in claim 1 in which the leading surface includes a level section between the upward incline and the top edge.

4. A method of mixing fluid as claimed in claim 3 in which a further upward incline extends between the level section and the top edge.

5. A method of mixing fluid as claimed in claim 1 in which the or each upward incline is at an angle of between about 5° and 45° from a bottom surface of the raceway channel.

6. A method of mixing fluid as claimed in claim 5 in which the or each upward incline an angle of between about 7° and 30° from the bottom surface of the raceway channel.

7. A mixing element for a raceway channel in which a fluid flows and in which the level of the fluid in the channel is at a substantially constant level, comprising a ramp which is configured to extend, in use, across the channel and has a leading surface which terminates in a top edge and a trailing surface which extends vertically downwardly from the top edge, the leading surface further having an upward incline extending at least partway along its length, wherein the height of the ramp is selected so that the top edge is operatively about one critical depth below the level of the fluid in the channel.

8. (canceled)

9. A mixing element as claimed in claim 7 in which the leading surface includes a level section between the upward incline and the top edge.

10. A mixing element as claimed in claim 9 in which a further upward incline extends between the level section and the top edge.

11. A mixing element as claimed in claim 7 in which the or each upward incline is at an angle of between about 5° and 45° from a bottom surface of the raceway channel.

12. A mixing element as claimed in claim 11 in which the or each upward incline an angle of between about 7° and 30° from the bottom surface of the raceway channel.

13. A raceway channel which forms an endless loop and has a pair of sides extending from a bottom and having a ramp which is configured to extend, in use, across the channel, the ramp having a leading surface which terminates in a top edge and a trailing surface which extends vertically downwardly from the top edge, the leading surface further having an upward incline extending at least partway along its length, wherein the height of the ramp is selected so that the top edge is operatively about one critical depth below the level of the fluid in the channel.

14. (canceled)

15. A raceway channel as claimed in claim 13 in which the leading surface includes a level section between the upward incline and the top edge.
